(19) United States
(12) Patent Application Publication
(10) Pub. No.: US 2018/0282679 A1
(43) Pub. Date: Oct. 4, 2018

(54) CELL CULTURE APPARATUS

(71) Applicant: The Ritsumeikan Trust, Kyoto-shi, Kyoto (JP)

(72) Inventors: Satoshi KONISHI, Kusatsu-shi, Shiga (JP); Takuya FUJITA, Kusatsu-shi, Shiga (JP); Koji HATTORI, Kusatsu-shi, Shiga (JP); Yusuke KONO, Kusatsu-shi, Shiga (JP)

(73) Assignee: The Ritsumeikan Trust, Kyoto-shi, Kyoto (JP)

(21) Appl. No.: 15/747,449

(22) PCT Filed: Jul. 26, 2016
(86) PCT No.: PCT/JP2016/071879

§ 371 Date: Jun. 20, 2018

(30) Foreign Application Priority Data
Jul. 28, 2015 (JP) 2015-148789

(51) Int. Cl. C12M 1/02; C12M 1/12

(52) U.S. Cl. CPC C12M 27/00; C12M 23/06; C12M 23/04

(57) ABSTRACT

Provided is a cell culture apparatus capable of constructing cultured cells under an environment closer to that in a living body. The cell culture apparatus includes: a culture substrate (11) having a culture surface (11a) on which cells S are cultured; and a drive unit (12) configured to allow opening and closing of the culture substrate (11) between a closed form and an open form. The closed form is a form in which the culture substrate (11) forms a flow path having an internal volume with the culture surface (11) being an internal surface of the flow path. The open form is a form in which the culture surface (11a) of the culture substrate (11) is opened outward more than the culture surface (11a) of the culture substrate (11) in the closed form.

### TECHNICAL FIELD

The present invention relates to an apparatus for artificially culturing cells of organisms.

### BACKGROUND ART

For example, in a process of designing a drug, artificially cultured cells of a living body are used for drug discovery screening performed for selecting candidate compounds.

To date, culturing of such cells has generally been performed on dishes or in microwells. In recent years, an organ-on-a-chip has been developed, which imitates an organ by culturing cells in a condition closer to a living body (refer to Patent Literature 1, for example).

### CITATION LIST

### Patent Literature

PATENT LITERATURE 1: Japanese Laid-Open Patent Publication No. 2014-506801

### SUMMARY OF INVENTION

### Technical Problem

When drug discovery screening is performed as described above, it is advantageous to use cultured cells that imitate a substance absorbing process in an intestine, having the longest residence time in a body.

However, when cells are two-dimensionally cultured on dishes or in microwells, a reaction that occurs in a living body cannot always be reproduced. For example, even if a drug solution is caused to flow over two-dimensionally cultured cells on the assumption that a liquid containing a drug (drug solution) flows in an intestine, the fluid does not uniformly act over the entirety of the cells, and a mechanical force such as a shearing force caused by the flow of the fluid cannot be appropriately applied to the cells. Thus, it is difficult to accurately reproduce the process in which the drug is absorbed in and transmitted through the cells.

Further, in the technique disclosed in Patent Literature 1, a mechanical force along a planar direction is applied to the cells planarly cultured on a flexible porous membrane, to imitate peristaltic movement of an intestine or the like and reproduce an environment in a living body. However, since this technique is similar to the aforementioned technique in that the cells are two-dimensionally cultured, it is difficult to reproduce a mechanical force caused by flow of a fluid.

An objective of the present invention is to provide a cell culture apparatus capable of constructing cultured cells under an environment closer to that in a living body.

### Solution to Problem

(1) A cell culture apparatus according to the present invention includes:

a culture substrate having a culture surface on which cells are cultured; and

a drive unit configured to allow opening/closing motion of the culture substrate between a closed form and an open form, wherein

the closed form is a form in which the culture substrate forms a flow path having an internal volume with the culture surface being an internal surface, and

the open form is a form in which the culture surface of the culture substrate is opened more than the culture surface of the culture substrate in the closed form.

In the cell culture apparatus having the above configuration, the culture substrate is opened and closed between the closed form in which the culture substrate forms a flow path having an internal volume with the culture surface being an internal surface of the flow path, and the open form in which the culture surface of the culture substrate is opened outward as compared to the culture surface of the culture substrate in the closed form. Therefore, by causing a drug solution or the like to flow in the flow path inside the culture substrate in the closed form, a force caused by the flow of the fluid can be reproduced under an environment closer to that in a living body. Accordingly, a reaction or the like that occurs in the cultured cells can be evaluated in a manner near to evaluation for a reaction or the like that occurs in cells of an actual living body. Further, since the culture surface of the culture substrate in the open form is opened more than the culture surface of the culture substrate in the closed form, cultivation and observation of the cells on the culture surface can be easily performed.

(2) The culture substrate in the open form is preferably flat in shape.

Thus, cultivation, observation, and the like of the cells can be performed in the same manner as the conventional manner.

(3) The culture substrate in the closed form is preferably tubular in shape.

This configuration allows an organ of a living body, such as an intestine, to be imitated, and allows a reaction of the organ when a drug solution or the like is applied thereto to be faithfully reproduced. Here, the term “tubular” is not limited to a form in which the entire outer periphery of the culture substrate is closed, but may include a form in which a portion of the outer periphery is opened.

(4) The drive unit preferably has a balloon actuator provided on a surface, of the culture substrate, on a side opposite to the culture surface.

Thus configuration allows the structure of the drive unit to be simplified.

(5) The balloon actuator may have a region through which a liquid having transmitted through the culture substrate passes.

In this configuration, the liquid having transmitted through the culture substrate passes through the balloon actuator of the drive unit to be discharged to the outside of the culture substrate. Therefore, it is possible to evaluate the state after, for example, a drug solution or the like flowing in the culture substrate in the closed form is absorbed in and transmitted through the cells.

(6) In the case where the culture substrate in the closed form is tubular in shape, culture-substrate-side surfaces of both end portions, opposing each other, of the balloon actuator are preferably seal surfaces that come into surface-contact with each other.

This configuration prevents leakage of a drug solution or the like that is caused to flow in the tubular culture substrate.

(7) Hydrophobic treatment is preferably applied to the seal surfaces.

This configuration reliably prevents leakage of a drug solution or the like that is caused to flow in the tubular culture substrate.

(8) Hydrophobic treatment is preferably applied to an inner peripheral surface of an end portion of the culture substrate in the closed form, to which an introduction tube for causing a fluid to flow into the culture substrate is connected.

This configuration inhibits backflow of a liquid from the culture substrate to the introduction tube.

### Advantageous Effects of Invention

The cell culture apparatus of the present invention allows construction of cultured cell under an environment closer to that in a living body.

### BRIEF DESCRIPTION OF DRAWINGS

FIGS. 1A and 1B illustrate schematic perspective views of a cell culture apparatus according to a first embodiment.

FIG. 2 is a cross-sectional view of a part of a culture substrate in an enlarged manner.

FIG. 3 is an explanatory diagram showing a balloon actuator.

FIGS. 4A and 4B illustrate explanatory diagrams illustrating an operation principle of the balloon actuator.

FIGS. 5A and 5B illustrate plan views illustrating a state where the cell culture apparatus is set in a fluid supply apparatus.

FIG. 6 is a cross-sectional view of the fluid supply apparatus.

FIG. 7 is an explanatory cross-sectional view showing a connection portion between an introduction tube and a culture substrate.

FIG. 8 is an explanatory plan view showing a balloon actuator according to a second embodiment.

FIGS. 9A and 9B illustrate explanatory diagrams illustrating an operation principle of a balloon actuator.

FIG. 10 is an explanatory plan view showing a balloon actuator according to a third embodiment.

FIGS. 11A and 11B illustrate cross-sectional views of a cell culture apparatus according to a fourth embodiment.

FIGS. 12A and 12B illustrate cross-sectional views of a cell culture apparatus according to a fifth embodiment.

FIGS. 13A and 13B illustrate cross-sectional views of a cell culture apparatus according to a sixth embodiment.

FIGS. 14A and 14B illustrate perspective views of a cell culture apparatus according to a seventh embodiment.

FIGS. 15A, 15B, 15C, 15D, 15E and 15F show images of a surface of a culture substrate in an evaluation test 1.

FIGS. 16A, 16B, 16C, 16D, 16E and 16F show images of a surface of a culture substrate in an evaluation test 2.

FIGS. 17A, 17B, 17C, 17D, 17E and 17F show images of a culture substrate and a balloon actuator used in an evaluation test 3.

FIG. 18 is a graph showing a relationship between an inner diameter of the culture substrate and a shearing stress that acts on a surface of the culture substrate in the evaluation test 3.

FIGS. 19A, 19B and 19C show images of the surface of the culture substrate in the evaluation test 3.

FIGS. 20A, 20B and 20C show images of the surface of the culture substrate in the evaluation test 3.

FIGS. 21A and 21B illustrates explanatory schematic diagrams illustrating a state where a drug is absorbed in cultured cells.

FIG. 22 is a table showing dynamic characteristics of a fluid.

### DESCRIPTION OF EMBODIMENTS

Hereinafter, specific embodiments of a cell culture apparatus will be described.

### First Embodiment

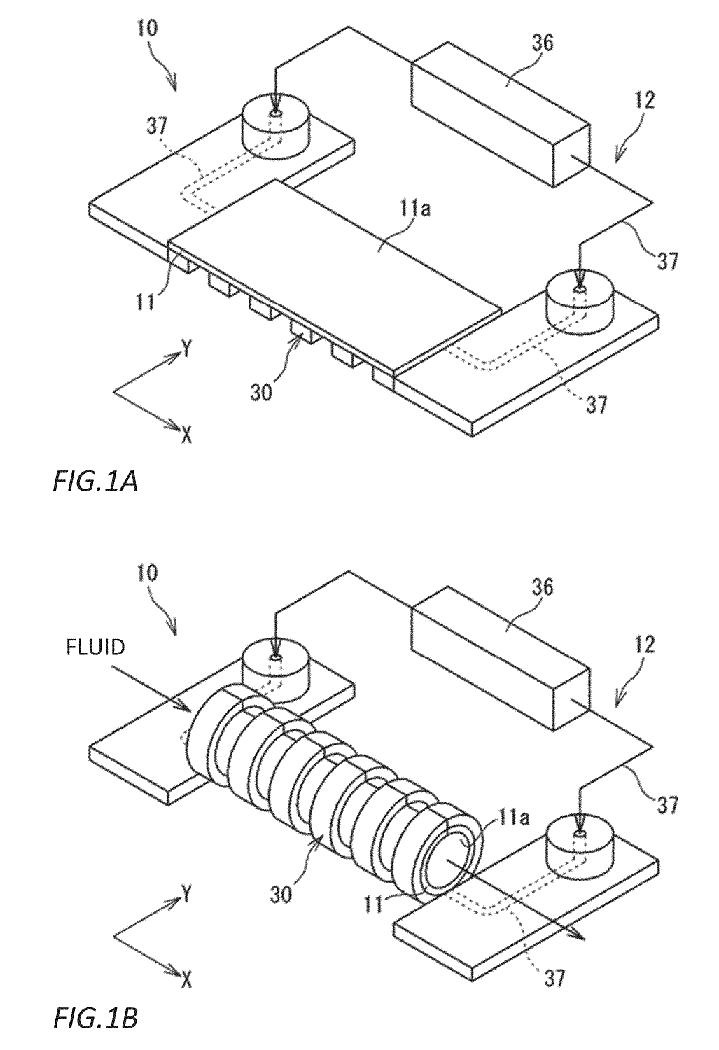

FIGS. 1A and 1B illustrate schematic perspective views of a cell culture apparatus according to a first embodiment.

The cell culture apparatus 10 of the present embodiment imitates an organ formed in a tubular shape by using artificially cultured cells, and a fluid such as a drug solution is caused to flow in the imitated organ to reproduce a reaction of the cultured cell in the organ. Examples of the tubular organ include an intestine, a blood vessel, and the like. In particular, the cell culture apparatus 10 of the present embodiment imitates an intestine as an example of a tubular organ. Further, the cell culture apparatus 10 of the present embodiment is able to perform evaluation for absorbency of a drug or the like by the cultured cells, and evaluation for permeability. However, the cell culture apparatus 10 of the present embodiment can also be used for independently performing evaluation for only one of absorbency and permeability.

The cell culture apparatus 10 of the present embodiment includes a culture substrate 11, and a drive unit 12 that applies a driving force to the culture substrate 11 to transform the culture substrate 11.

The culture substrate 11 is formed in a rectangular shape in a plan view. An upper surface of the culture substrate 11 is a culture surface 11a on which cells are seeded and cultured. The culture substrate 11 is elastically transformable.

The drive unit 12 allows opening/closing motion of the culture substrate 11. Specifically, the culture substrate 11 is transformed between a planar (flat) form (also referred to as an open form) as shown in FIG. 1A and a tubular (cylindrical) form (also referred to as a closed form) as shown in FIG. 1B. Regarding the tubular form, the culture substrate 11 is formed in a tubular shape with the culture surface 11a being an inner surface, and allows a fluid to flow therein as indicated by an arrow. In other words, the culture substrate 11 in the tubular form forms a flow path having an internal volume with the culture surface 11a being an inner surface of the flow path. Meanwhile, the culture substrate 11 in the planar form has the culture surface 11a widely opened outward as compared to the culture substrate 11 in the tubular form. That is, since the culture substrate 11 in the tubular form is tubular in shape with the outer periphery thereof being completely closed, the culture surface 11a is opened to the outside only at the both ends in the tube-axis direction, whereas the culture substrate 11 in the planar form is in the state where the culture surface 11a thereof is entirely opened.

The culture substrate 11 takes the tubular form when a driving force from the drive unit 12 is applied thereto, and takes the planar form when the driving force is canceled.

In the following description, the tube-axis direction (cylinder-axis direction) of the culture substrate 11 in the tubular form is an X direction, and a horizontal direction orthogonal to the X direction is a Y direction. Therefore, each of the sides of the culture substrate 11 in the planar form is arranged in parallel to the X direction or the Y direction.

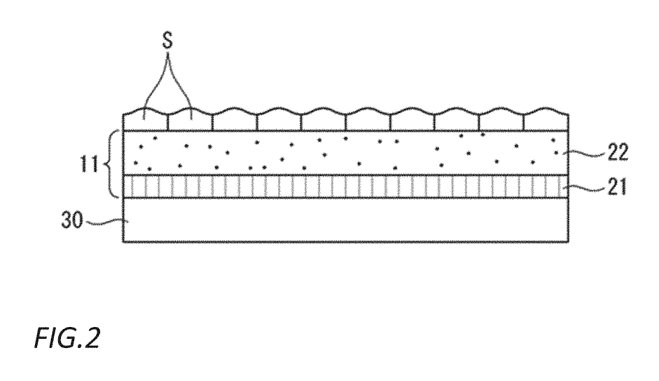

FIG. 2 is an explanatory cross-sectional view showing a part of the culture substrate 11 in an enlarged manner. The culture substrate 11 includes a filter 21 provided on the drive unit 12 (a balloon actuator 30 described later), and a collagen sheet 22 serving as an extracellular matrix (ECM) provided on the filter 21. Cells S are cultured on the collagen sheet 22 with the culture substrate 11 being immersed in a liquid serving as a culture medium. In the present embodiment, as the cells S, caco-2 cells are statically cultured on the culture substrate 11 to imitate an intestinal epithelium. The filter 21 is a porous film that allows, for example, a liquid containing a drug to pass therethrough. As for the filter 21, a filter that can be joined to a later-described balloon actuator 30 (made of PDMS) of the drive unit 12, for example, a filter formed of a material such as polycarbonate (PC), polymethyl methacrylate (PMMA), or polypropylene (PP), can be used. The culture substrate 11 may be configured by providing an ECM directly on the drive unit 12 (balloon actuator 30).

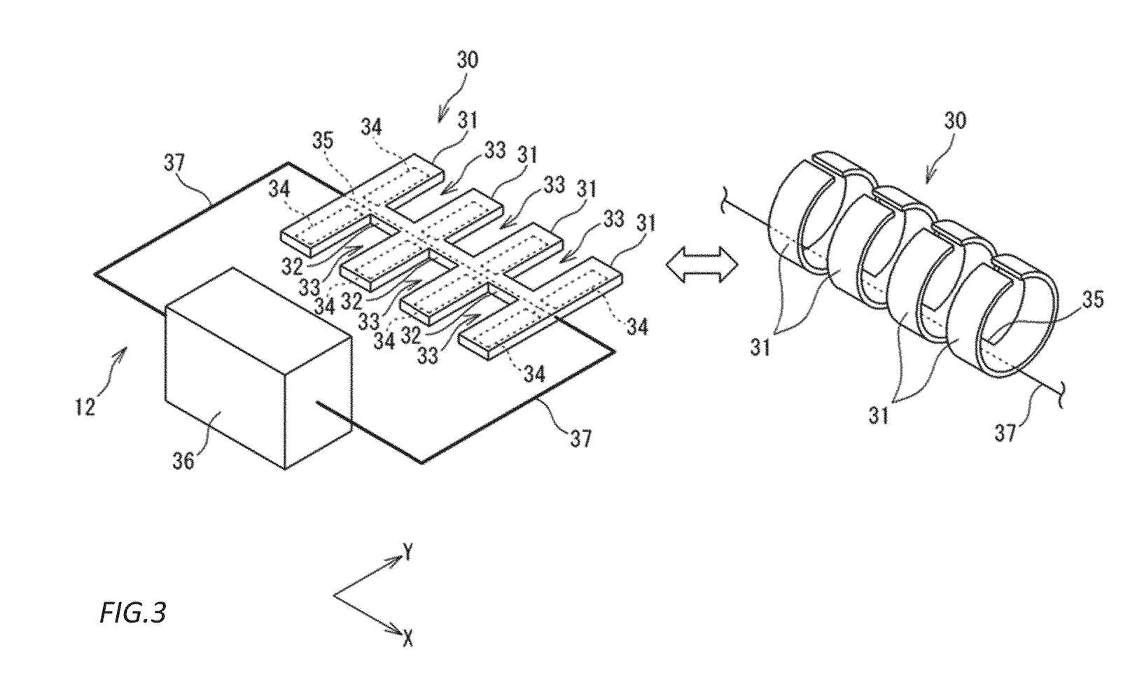

FIG. 3 is an explanatory diagram showing the drive unit 12.

The drive unit 12 includes: the balloon actuator 30; an air supply unit 36 that supplies air to the balloon actuator 30; and a supply pipe 37 that connects the balloon actuator 30 to the air supply unit 36, and serves as an air supply path from the air supply unit 36 to the balloon actuator 30. The balloon actuator 30 is elastically transformed in accordance with a change in the internal air pressure, and causes the culture substrate 11 to perform a predetermined action. The balloon actuator 30 of the present embodiment includes a plurality of local actuators 31 arranged in the X direction, and each local actuator 31 is formed into an elongated shape in the Y direction. The plurality of local actuators 31 are connected to each other by a connection part 32 at center portions thereof in the Y direction so as to be integrated. Between adjacent local actuators 31, a transmission region 33 is formed in a notch shape from an outer edge toward the inside in the Y direction. Therefore, the liquid transmitted through the culture substrate 11 can pass through the balloon actuator 30 via the transmission regions 33.

Each local actuator 31 has hollow portions 34 therein at both sides in its length direction (Y direction). The hollow portions 34 are communicated with each other via an internal tube 35. The internal tube 35 is connected to the air supply unit 36 such as a compressor via the supply pipe 37. When the air supply unit 36 supplies air to the hollow portions 34 of each local actuator 31, both end portions, in the longitudinal direction, of the local actuator 31 approach each other, whereby the local actuator 31 transforms from a planar shape to a ring shape (refer to the right side in FIG. 3). By maintaining the state where air is being supplied to the hollow portions 34, each local actuator 31 is kept in the ring shape. When the air supply unit 36 stops supply of air to the hollow portions 34, each local actuator 31 returns to the planar form due to elastic recovery (refer to the left side in FIG. 3).

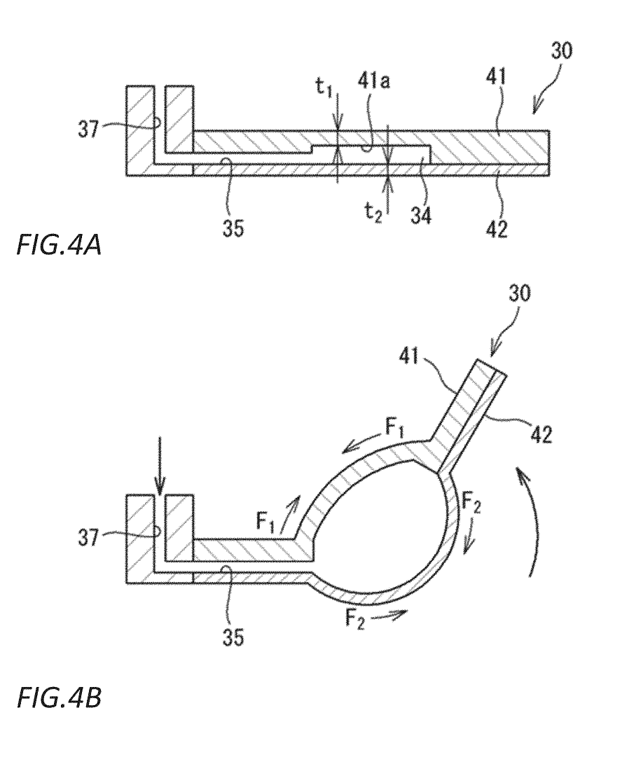

FIGS. 4A and 4B illustrate explanatory cross-sectional views illustrating an operation principle of the balloon actuator.

The balloon actuator 30 (each local actuator 31) is composed of two layers of silicone rubber, including a first film body 41 and a second film body 42. A hollow portion 34 is formed between the first film body 41 and the second film body 42. The hollow portion 34 is configured by forming a recess 41a at one or both of opposing surfaces of the first and second film bodies 41 and 42.

Each of the first film body 41 and the second film body 42 is formed of a PDMS (polydimethylsiloxane) thin film that is a kind of silicone rubber. The first film body 41 and the second film body 42 have different thicknesses. Specifically, a thickness t₁ of a portion of the first film body 41, where the recess 41a is formed, is greater than a thickness t₂ of the second film body 42. The first film body 41 and the second film body 42 have different hardnesses. Specifically, the first film body 41 is formed so as to have a higher hardness than the second film body 42.

The first film body 41 and the second film body 42 are formed of silicone rubber which is retractable, and therefore are extended and expanded like a balloon while increasing the surface areas thereof, by the pressure of air supplied into the hollow portion 34. Since, in the balloon actuator 30, the second film body 42 is thinner and softer than the first film body 41, the second film body 42 is expanded more than the first film body 41 under the same pressure.

When the first film body 41 and the second film body 42 are expanded, tensile stresses F₁ and F₂ are generated in the respective film bodies 41 and 42 as shown in FIG. 4B. Since the first film body 41 is thicker and harder than the second film body 42, the tensile stress F₁ in the first film body 41 is greater than the tensile stress F₂ in the second film body 42. Therefore, in the balloon actuator 30, bending motion in the same direction as the expanding direction of the first film body 41, that is, upward bending motion, occurs in the example of FIG. 4B. Two or more recesses 41a may be formed in the longitudinal direction of each local actuator 31.

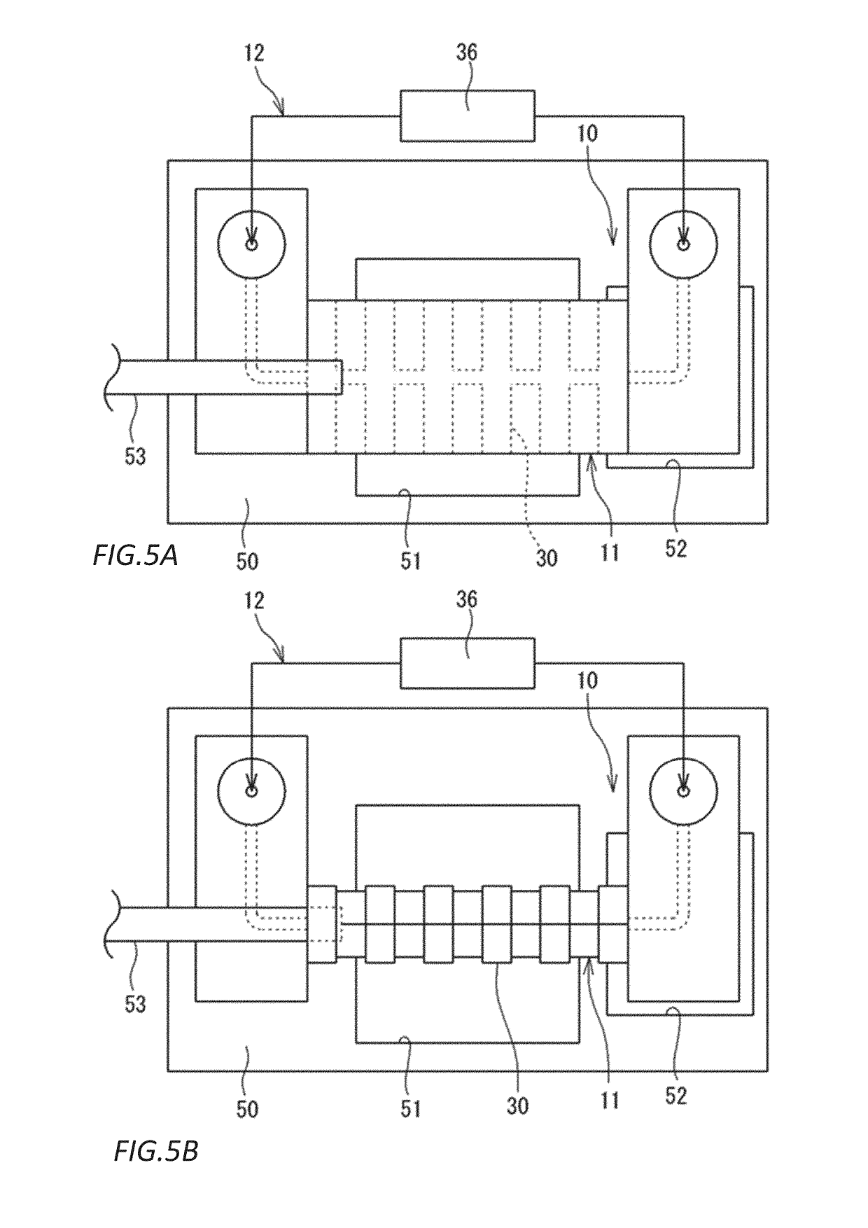

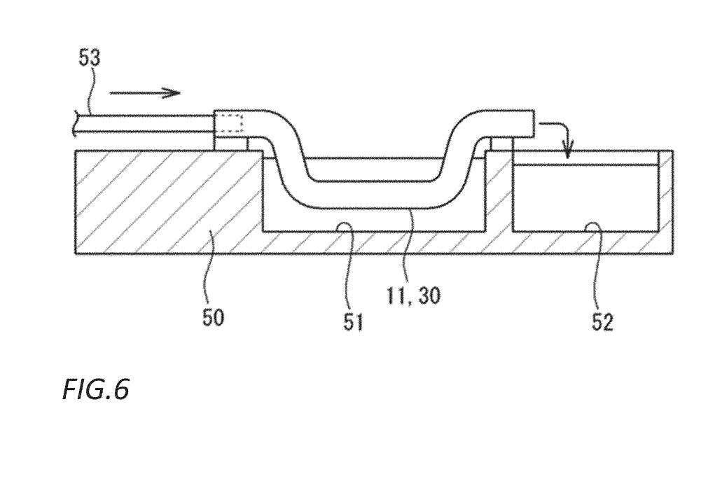

FIGS. 5A, 5B and 6 each show an apparatus for causing a fluid to flow in the culture substrate 11 of the cell culture apparatus 10 shown in FIGS. 1A and 1B. The cell culture apparatus 10 is placed on a storage container 50. FIG. 5A shows a state where the culture substrate 11 is in the planar form, and FIG. 5B shows a state where the culture substrate 11 is in the tubular form. The storage container 50 has a first pool (first storage portion) 51 and a second pool (second storage portion) 52 formed therein. A liquid is stored in the first pool 51 in advance. As for this liquid, a culture medium used when cells are cultured, for example, DMEM (Dulbecco-modified eagle's medium), can be used. The culture substrate 11 in the tubular form is immersed in the liquid stored in the first pool 51. In FIG. 6, the culture substrate 11 and the balloon actuator 30 are sharply bend at the front and rear of the first pool 51. However, actually, the culture substrate 11 and the balloon actuator 30 are smoothly curved when being immersed in the first pool 51. As for the liquid stored in the first pool 51, a saline solution such as BS (Buffered Saline) or HBSS (Hank's Balanced Salt Solution) may be used.

An introduction tube 53 is connected to one of end portions of the culture substrate 11 in the tubular form. For example, a drug solution containing a drug to be subjected to drug discovery screening flows through the introduction tube 53, and is perfused into the culture substrate 11 in the tubular form. The other end portion of the culture substrate 11 faces the second pool 52, and the drug solution that flows out from the inside of the culture substrate 11 is stored in the second pool 52.

Since the culture substrate 11 has the tubular form, a mechanical force such as a shearing force caused by the flow of the drug solution acts on the cultured cells on the inner surface of the culture substrate 11, in a manner close to that in an intestine of an actual living body. Then, the drug solution perfused in the culture substrate 11 is absorbed in the cultured cells, and transmits through the filter 21 to ooze out of the culture substrate 11. Then, the drug solution passes through the transmission regions 33 of the balloon actuator 30, and is mixed with the liquid inside the first pool 51. Accordingly, permeability of the drug can be appropriately evaluated based on the state of the liquid stored in the first pool 51. Further, since the drug solution stored in the second pool 52 may contain, for example, a component secreted from the cultured cells due to absorption of the drug, this drug solution can also be evaluated.

After the drug solution is perfused into the culture substrate 11 in the tubular form, the culture substrate 11 is transformed to the planar form as shown in FIG. 5A, whereby the state of the cultured cells that have absorbed the drug and reacted with the drug, can be easily observed.

Further, the operation of circulating the drug solution through the culture substrate in the tubular form and thereafter observing the cells on the culture substrate in the planar form can be repeatedly performed.

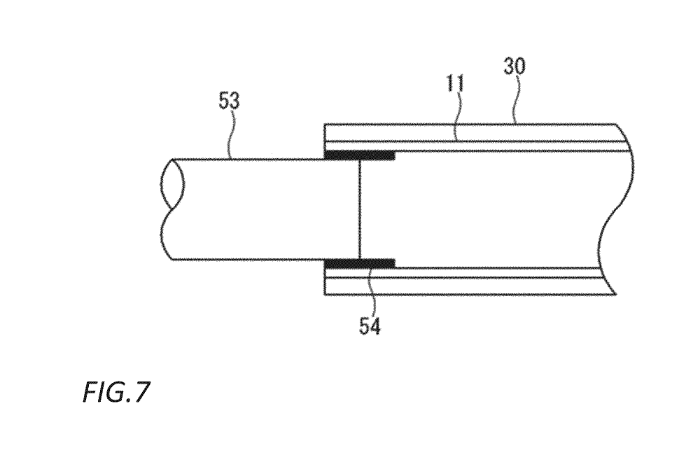

As shown in FIG. 7, hydrophobic treatment is applied to the inner surface of an end portion, in the axial direction, of the culture substrate 11 in the tubular form, to which the introduction tube 53 is connected. For example, the inner surface of the end portion of the culture substrate 11 is coated with a hydrophobic film 54 such as a parylene film. Therefore, the drug solution that flows into the culture substrate 11 through the introduction tube 53 can be prevented from flowing back into the introduction tube 53 and from leaking.

### Second Embodiment

In the first embodiment described above, the culture substrate 11 is transformed from the planar form to the tubular form when the driving force from the balloon actuator 30 acts thereon, and the culture substrate 11 is transformed from the tubular form to the planar form due to elastic recovery when the driving force from the balloon actuator 30 is canceled. In this second embodiment, the driving force from the balloon actuator 30 is applied even when the culture substrate 11 is transformed from the tubular form to the planar form.

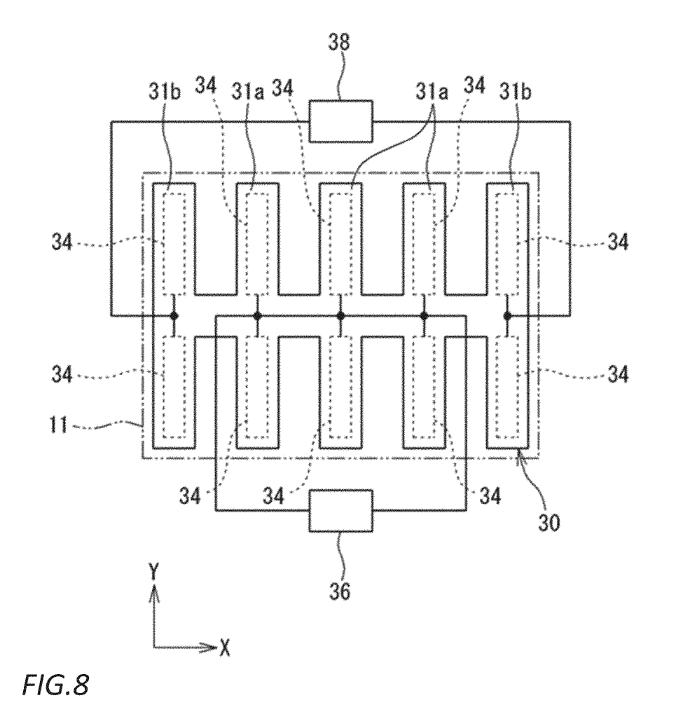

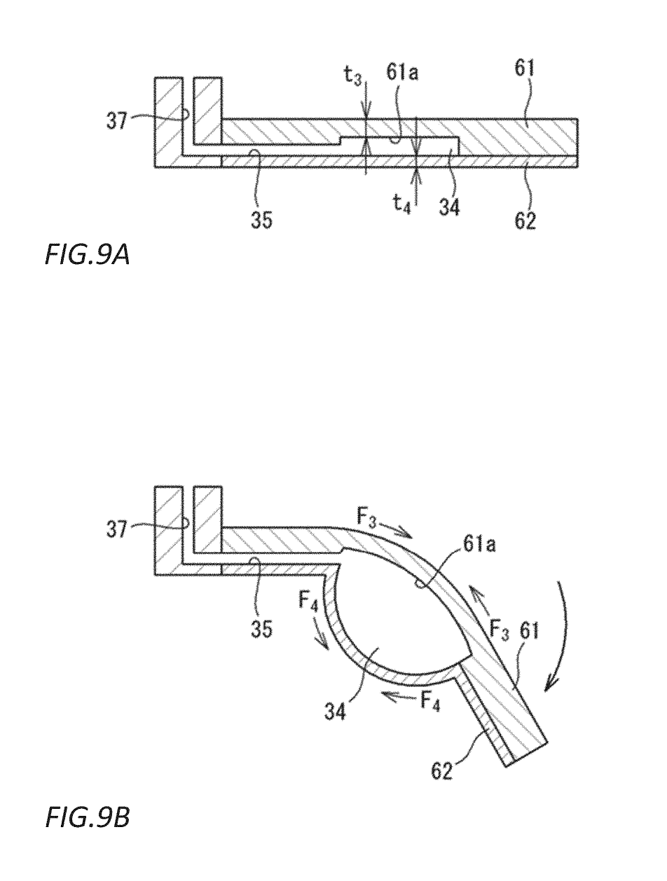

Specifically, as shown in FIG. 8, the balloon actuator 30 includes two types of local actuators 31a and 31b. Each of the local actuators 31a is similar to the local actuator of the first embodiment, and performs upward bending motion when air is supplied to the hollow portion 34 as shown in FIG. 4B. On the other hand, each of the local actuators 31b performs downward bending motion when air is supplied to the hollow portion 34 as shown in FIG. 9B. One local actuator 31b is disposed at each of the both sides in the X direction, while a plurality of local actuators 31a are disposed between the local actuators 31b at the both ends.

As shown in FIGS. 9A and 9B, each local actuator 31b consists of a third film body 61 and a fourth film body 62, and both the film bodies 61 and 62 are formed of PDMS. A thickness t₃ of a portion of the third film body 61, where a recess 61a is formed, is greater than a thickness t₄ of the fourth film body 62. The third film body 61 has a hardness higher than that of the fourth film body 62. In this regard, the relationship between the third film body 61 and the fourth film body 62 is the same as the relationship between the first film body 41 and the second film body 42 of the local actuator 31a. However, the thickness t₃ of the third film body 61 of the local actuator 31b is greater than the thickness t₁ (refer to FIG. 4A) of the first film body 41 of the local actuator 31a, and the hardness thereof is higher than that of the first film body 41. Further, air is supplied to the local actuators 31b from an air supply unit 38 (refer to FIG. 8) different from that for the local actuators 31a.

In the local actuator 31b, when air is supplied to the hollow portion 34 thereof, the third film body 61 is hardly extended, and a tensile stress F₃ generated in the third film body 61 is small. On the other hand, the fourth film body 62 is greatly extended and expanded, whereby a tensile stress F₄ greater than the tensile stress F₃ generated in the third film body 61 is generated in the fourth film body 62, which causes downward bending motion of the local actuator 31b.

As for the balloon actuator 30 of the present embodiment, when the culture substrate 11 is transformed from the planar form to the tubular form, only the air supply unit 36 is operated to cause the local actuators 31a to perform bending motion. When the culture substrate 11 is transformed from the tubular form to the planar form, only the air supply unit 38 is operated to cause the local actuators 31b to perform bending motion. Therefore, not only in transformation from the planar form to the tubular form but also in transformation from the tubular form to the planar form, the driving force from the balloon actuator 30 acts on the culture substrate 11, thereby realizing rapid transformation.

The local actuators 31b are provided only at the both ends of the culture substrate 11 in the X direction, and the local actuators 31a are provided on the most part of the culture substrate 11. Therefore, a greater driving force can be applied when the culture substrate 11 is transformed from the planar form to the tubular form, whereby the culture substrate 11 can be transformed to the tubular form more rapidly, and the tubular form can be reliably maintained.

Since the third film body 61, of the local actuators 31b, on the culture substrate 11 side is hardly expanded, the third film body 61 does not adversely affect the cells cultured on the culture substrate 11.

### Third Embodiment

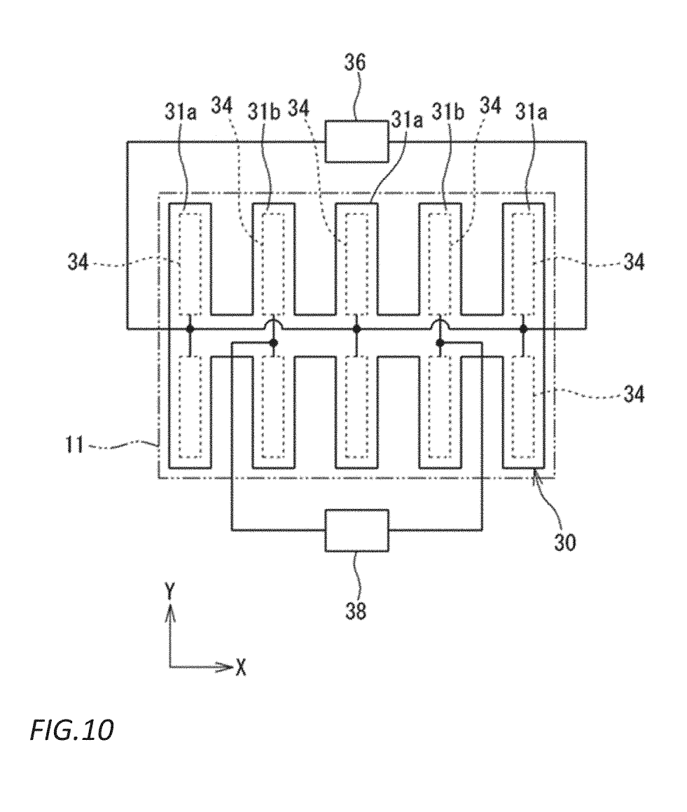

FIG. 10 is an explanatory plan view showing a balloon actuator according to a third embodiment. The balloon actuator 30 of this embodiment includes two types of local actuators 31a and 31b as in the second embodiment, and the local actuators 31a and the local actuators 31b are alternately arranged in the X direction.

Therefore, in the present embodiment, in both cases where the culture substrate 11 is transformed from the planar form to the tubular form and where the culture substrate 11 is transformed from the tubular form to the planar form, a driving force can be applied to the culture substrate 11 in a well-balanced manner.

In the second and third embodiments, a local actuator obtained by reversing the front and back surfaces of the local actuator 31a may be used as the local actuator 31b. In this case, the local actuator 31b performs bending motion in the opposite direction (downward direction) according to the same operation principle as shown in FIGS. 4A and 4B, whereby a driving force can be applied to the culture substrate 11 when the culture substrate 11 is transformed from the tubular form to the planar form. In this case, however, since the second film body 42 that is more expanded is disposed so as to face the culture surface 11a, the local actuators 31b are preferably disposed in areas where the local actuators 31b are less likely to affect cell culturing, for example, the both end portions in the X direction as shown in FIG. 8.

### Fourth Embodiment

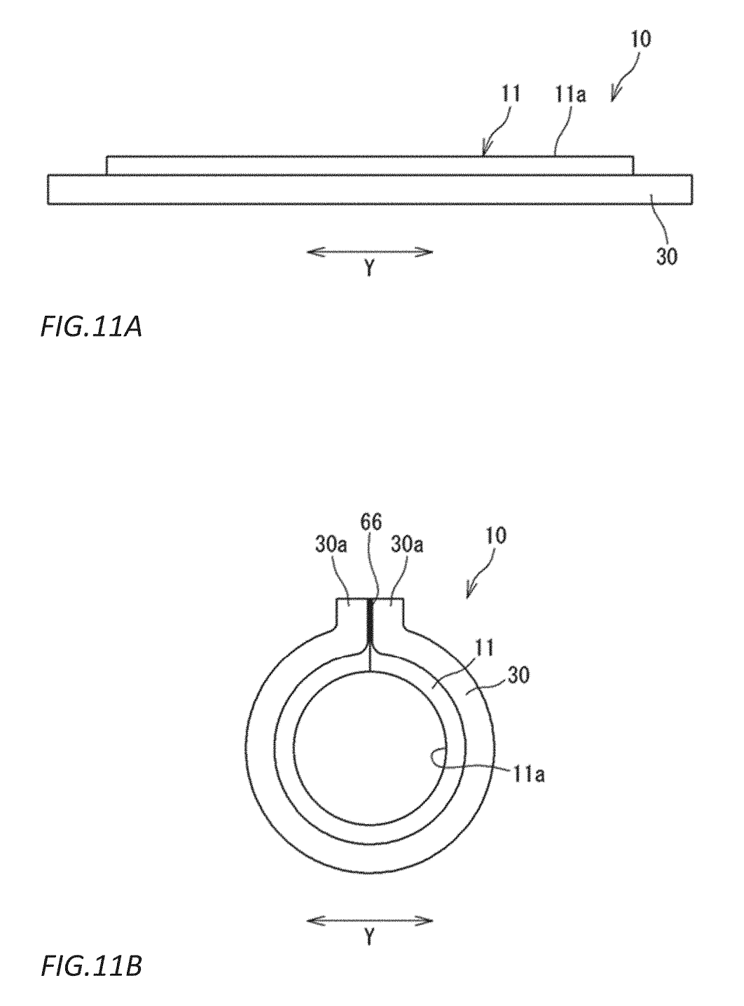

FIGS. 11A and 11B illustrate cross-sectional views of a cell culture apparatus according to a fourth embodiment.

In the present embodiment, as shown in FIG. 11A, the width of the balloon actuator 30 in the Y direction is greater than the width of the culture substrate 11. As shown in FIG. 11B, when the culture substrate 11 is transformed to the tubular form, both end portions 30a of the balloon actuator 30 in the Y direction protrude radially outward from the culture substrate 11 in the tubular form and are joined to each other to come into surface-contact with each other. This prevents leakage of a fluid when the fluid is caused to flow inside the culture substrate 11 in the tubular form.

Further, hydrophobic treatment is applied to the contact faces (seal faces) of the both end portions 30a of the balloon actuator 30. For example, a hydrophobic film 66 can be provided on the seal faces. Thus, leakage of the fluid that flows in the culture substrate 11 in the tubular form can be prevented more reliably. However, the hydrophobic treatment should not inhibit sticking of the seal faces. In the case where the balloon actuator 30 consists of a plurality of local actuators 31, 31a, and 31b as shown in FIG. 3, FIG. 9A, FIG.9B and FIG. 10, portions to be joined to each other may be formed at both end portions of the balloon actuator 30 in the Y direction so as to extend in the Y direction, and the plurality of local actuators 31, 31a, and 31b that are adjacent to each other in the X direction may be connected to each other at the extended portions.

### Fifth Embodiment

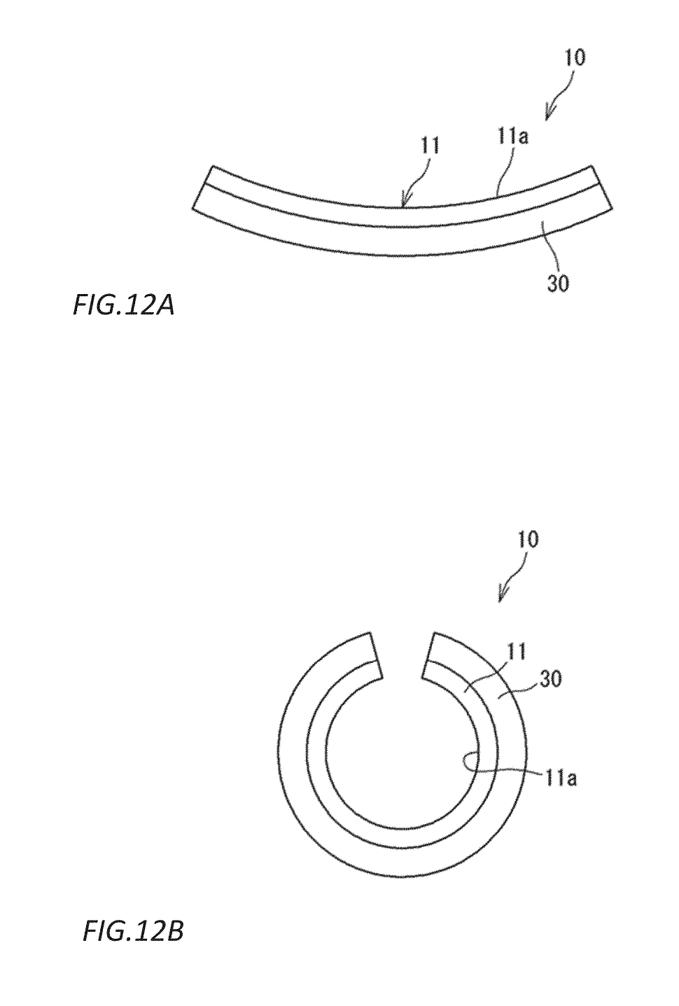

FIGS. 12A and 12B illustrates cross-sectional views of a cell culture apparatus according to a fifth embodiment.

In the present embodiment, the culture substrate 11 in the open form is curved in a circular arc shape as shown in FIG. 12A, and the culture substrate 11 in the closed form is curved with an arc radius smaller than that of the culture substrate 11 in the open form as shown in FIG. 12B.

The both end portions, in the Y direction, of the culture substrate 11 in the closed form are not in contact with each other, and a gap is formed between the both end portions. Also in such a closed form, the culture substrate 11 can be regarded as having a tubular shape, and therefore forms a flow path having an internal volume with the culture surface 11a being an inner surface of the flow path. Accordingly, a fluid can be perfused in the culture substrate 11 in the closed as in the first embodiment described above.

Although the culture substrate 11 in the open form is curved, since the culture surface 11a thereof is opened wider than the culture surface 11a in the open form, cultivation and observation of the cells on the culture surface 11a can be satisfactorily performed.

Also in this embodiment, the culture substrate 11 in the open form may be formed in a planar shape as in the first embodiment. On the other hand, the culture substrate 11 in the open form in the first embodiment may be slightly curved as in the present embodiment. The degree of curvature of the culture substrate 11 in the closed form is sufficient if it allows a liquid to be perfused in the culture substrate 11, but it is preferable that the culture substrate 11 is curved within a range exceeding 180° around the center axis of the tubular form.

### Sixth Embodiment

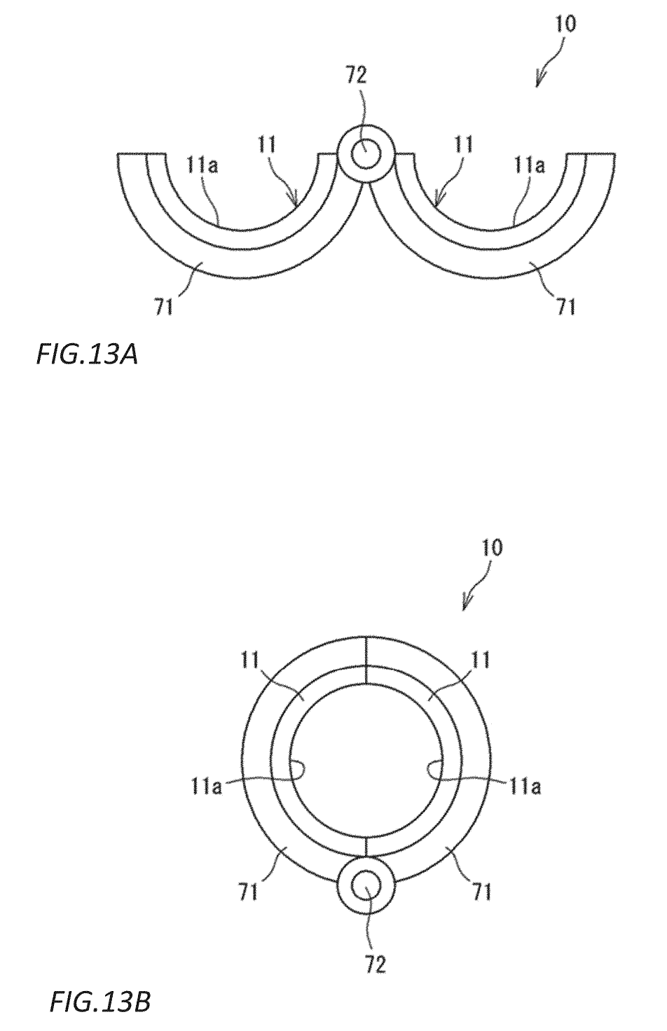

FIGS. 13A and 13B illustrate cross-sectional views of a cell culture apparatus according to a sixth embodiment.

In this embodiment, two support members 71 each having a semicircular arc-shape are provided, and a culture substrate 11 having a culture surface 11a is provided on an inner surface of each support member 71. One-end portions of the two support members 71 are pivotably connected to each other by a hinge 72. Each support member 71 has rigidity enough to maintain the semicircular arc-shape. As shown in FIG. 13B, the culture substrates 11 can be transformed to a closed form (tubular form) by bringing the other-end portions of the two support members 71 into contact with one another. As shown in FIG. 13A, the culture substrates 11 can be transformed to an open form by separating the other-end portions of the two support members 71 from each other.

Also in this embodiment, a flow path is formed by the culture substrates 11 in the closed form, and a fluid can be perfused in the flow path. Since the culture surfaces 11a of the culture substrates 11 in the open form are widely opened, cultivation and observation of the cells can be satisfactorily performed.

In the present embodiment, the culture substrates 11 can be transformed between the closed form and the open form by pivoting the two support members 71 using a drive unit (not shown) including a motor, a fluid pressure cylinder, and the like. The support members 71 can be formed of a synthetic resin material, for example. The support members 71 may have transmission regions that allow the liquid transmitted through the culture substrate 11 to pass therethrough, as in the first embodiment.

### Seventh Embodiment

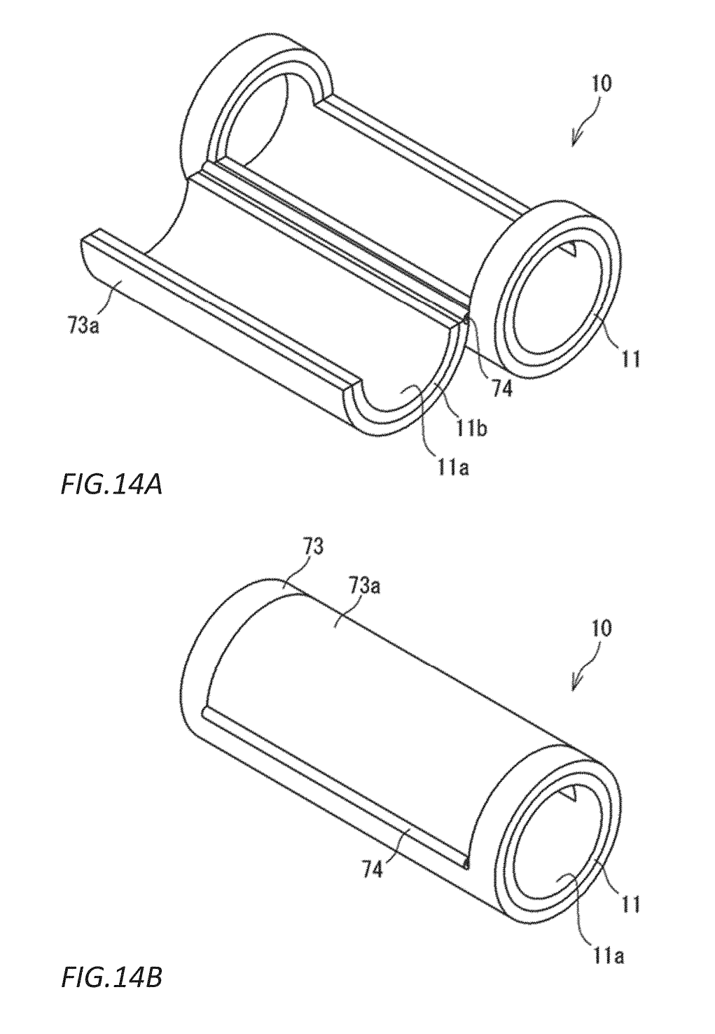

FIGS. 14A and 14B illustrate perspective views of a cell culture apparatus according to a seventh embodiment.

In this embodiment, as shown in FIG. 14B, a culture substrate 11 is provided inside a support member 73 formed in a tubular shape (cylindrical shape), and a flow path is formed inside the culture substrate 11. Further, in the present embodiment, a portion 73b of the support member 73 and a portion 11b of the culture substrate 11 inside the portion 73b are configured to be pivotable with respect to other portions via a hinge 74.

Therefore, opening/closing motion of the culture substrate 11 is allowed between the closed form shown in FIG. 14B and the open form shown in FIG. 14A, and the culture surface 11a of the culture substrate 11 in the open form is opened more than the culture surface 11a of the culture substrate 11 in the closed form. Therefore, cultivation and observation for the culture surface 11a of the culture substrate 11 in the open form can be satisfactorily performed.

Also in this embodiment, the culture substrate 11 can be transformed between the closed form and the open form by pivoting the portion 73b of the support member 73 by using a drive unit (not shown) including a motor, a fluid pressure cylinder, and the like. The support member 73 may have transmission regions that allows the liquid transmitted through the culture substrate 11 to pass therethrough, as in the first embodiment.

The present invention is not limited to the above embodiments, and changes may be made as appropriate within the scope of the present invention described in the claims.

For example, the drive unit 12 that transforms the culture substrate 11 is not limited to the structure using the balloon actuator 30, and may have any structure as long as it can transform the culture substrate 11 between the tubular form (closed form) and the planar form (open form).

The drive unit 12 may cause the culture substrate 11 to perform other motions than transformation between the closed form and the open form, for example, a motion that imitates peristaltic movement of a tubular organ. This motion can be realized by making the plurality of local actuators 31 of the first embodiment independently drivable, and causing the local actuators 31 to perform contraction motion in order in the X direction.

The cross-sectional shape of the culture substrate 11 in the tubular form need not be a perfect circle, and may be an ellipse or a flat circle.

Each of the transmission regions 33 of the balloon actuator 30 need not be formed in a notch shape, and may be formed in a hole shape penetrating the balloon actuator 30 in the thickness direction.

In a case where permeability of a liquid through the cultured cells is not evaluated, the balloon actuator 30 may be provided so as to cover the entire surface, of the culture substrate 11, on a side opposite to the culture surface 11a. In this case, liquid absorbency of the cultured cells can be evaluated.

### [Evaluation Test 1]

The inventors of the present application studied influences of the opening/closing motion of the culture substrate caused by the balloon actuator, on cells cultured on a culture substrate.

Specifically, caco-2 cells imitating intestinal epithelium were cultured on a culture substrate. Caco-2 cells produced by DS pharma biomedical Co., Ltd. were used. The caco-2 cells were cultured in DMEM (Dulbecco-modified eagle's medium) to which 10% heat inactivated fetal bovine serum, penicillin G (100 U mL⁻¹), streptomycin (100 μg mL⁻¹), and 1% non-essential amino acid were added, under an environment at 37° C., 5% CO), and 95% air. The caco-2 cells were dissociated in EDTA and 0.05% trypsin and passaged, and then the cells were seeded on collagen that forms a culture substrate on an upper surface of a balloon actuator. The caco-2 cells reached confluence in 7 days. The culture medium was replaced every 24 hours.

As for the balloon actuator, a balloon actuator as shown in FIGS. 11A and 11B was used in which both end portions of the culture substrate were joined to each other and sealed when the culture substrate was transformed to the tubular form. The balloon actuator did not have transmission regions that allowed liquids to pass therethrough, and externally covered the entire culture substrate.

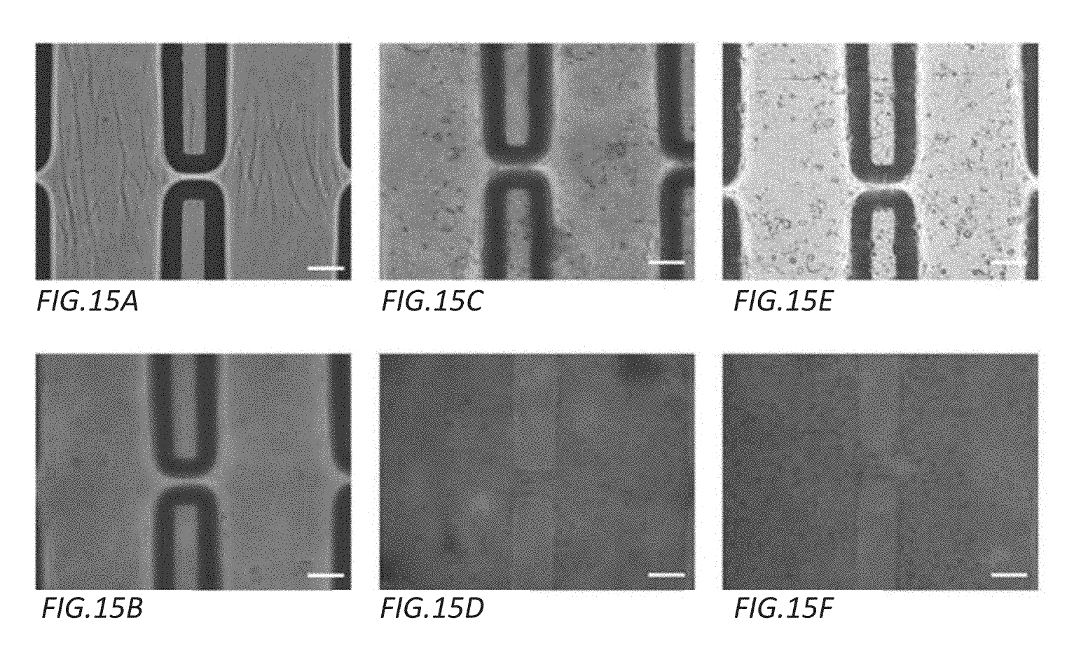

After a single layer of caco-2 cells was cultured on the culture substrate, the caco-2 cells were exposed to calcein AM (1 μg mL⁻¹) for 1 hour. Then, the balloon actuator was operated to transform the culture substrate from the planar form to the tubular form for 10 seconds, and bright field images and fluorescence images of the caco-2 cells before and after the operation of the balloon actuator were observed. This sequence was repeated 10 times. FIGS. 15A, 15B, 15C, 15D, 15E and 15F show the images of the surface of the culture substrate.

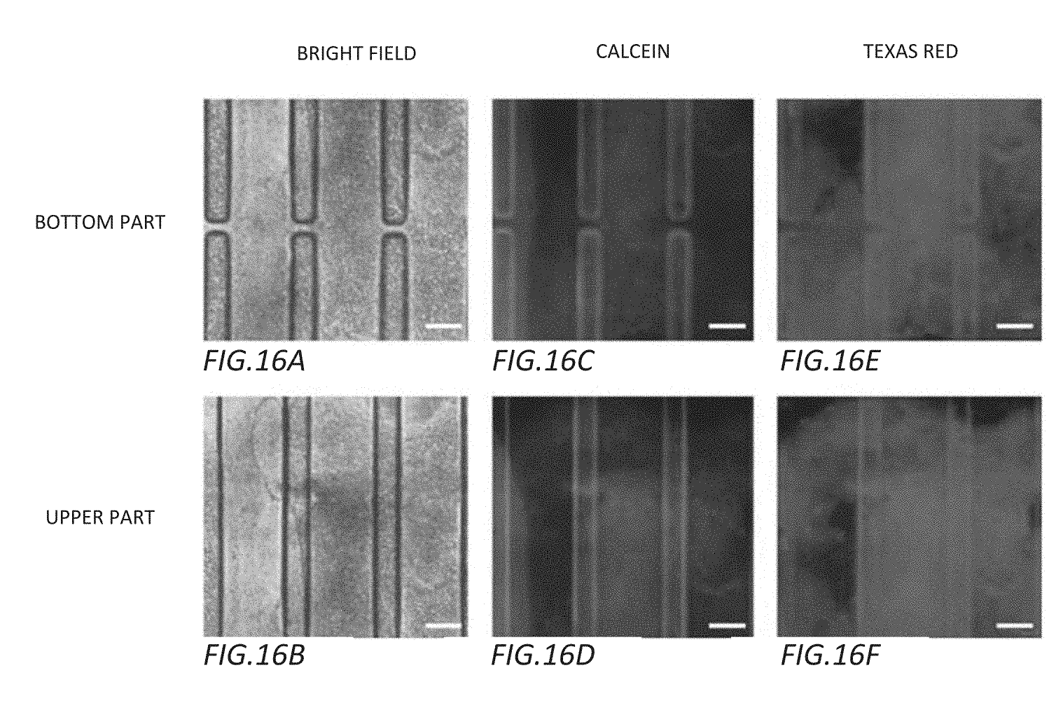

FIG. 15A shows the state after the upper surface of the balloon actuator has been coated with collagen, FIG. 15B shows the state after cells have been seeded, FIG. 15C shows the state after caco-2 cells have been statically cultured, FIG. 15D shows the state after the statically cultured caco-2 cells have been stained with calcein AM, FIG. 15E shows the state after the balloon actuator has been repeatedly operated 10 times, and FIG. 15F shows the state of the caco-2 cells stained with calcein AM after the balloon actuator has been repeatedly operated 10 times. In FIGS. 15A-15F and later-described FIGS. 16A-16F, each of U-shaped lines seen in the images shows the periphery of the hollow portion in the balloon actuator.

As a result of the above operation, it was confirmed that the caco-2 cells adhered to the surface of the culture substrate, and a single layer of caco-2 cells was normally formed on the culture substrate, as shown in FIG. 15B. In addition, it was confirmed that detachment of the caco-2 cells did not occur even after the balloon actuator was repeatedly operated 10 times (refer to FIGS. 15C and 15E). These results reveal that viability of the cells are maintained, and the caco-2 cells firmly adhere to the culture substrate even during the repeated operation of the balloon actuator. In addition, it was confirmed that the caco-2 cells on the culture substrate were uniformly stained by calcein AM (refer to FIGS. 15D and 15F). This reveals that the single layer of caco-2 cells is firmly formed by intercellular junctions. When a fluid was caused to flow in the culture substrate in the tubular form, leakage of the fluid did not occur.

### [Evaluation Test 2]

Next, the inventors of the present application studied whether a drug uniformly flowed in the culture substrate in the tubular form and was uniformly absorbed in the caco-2 cells.

The culture substrate in the tubular form was perfused with HBSS (Hanks' Balanced Salt Solution) containing fluorescent dyes, as a liquid corresponding to a drug solution, at a flow rate of 0.05 mL min⁻¹ for 1 hour. As for the fluorescent dyes, calcein as a model of a hydrophilic drug and Texas Red as a model of a lipophilic drug were used, and the concentrations thereof were set to 100 μmol L⁻¹ and 10 μmol L⁻¹, respectively. Thereafter, bright field images and fluorescence images of the caco-2 cells at a bottom part and an upper part of the culture substrate in the tubular form were observed.

FIGS. 16A, 16B, 16C, 16D, 16E and 16G show microscope images of the caco-2 cells. In particular, FIGS. 16A and 16B show bright field images at the bottom part and the upper part of the culture substrate in the tubular form, respectively. From these images, it was confirmed that detachment of the caco-2 cells did not occur due to a shearing stress associated with perfusion of the drug solution. FIGS. 16C and 16D show images obtained by imaging fluorescence signals from calcein at the bottom part and the upper part of the culture substrate in the tubular form, respectively. FIGS. 16E and 16F show images obtained by imaging fluorescence signals from Texas Red. From these images, it was confirmed that calcein and Texas Red were uniformly absorbed in the caco-2 cells regardless of the degree of hydrophilic property.

### [Evaluation Test 3]

The inventors of the present application studied dynamic characteristics of a fluid and absorption of a drug into a culture substrate in a tubular form when a liquid such as a drug solution was caused to flow in the culture substrate. Specifically, a fluid containing a drug was caused to flow in a plurality of types of tubular-form culture substrates having different inner diameters, and the states of the culture substrates before and after the flow of the fluid were observed. In addition, dynamic characteristics of the fluid in the respective tubular-form culture substrates were obtained by calculation.

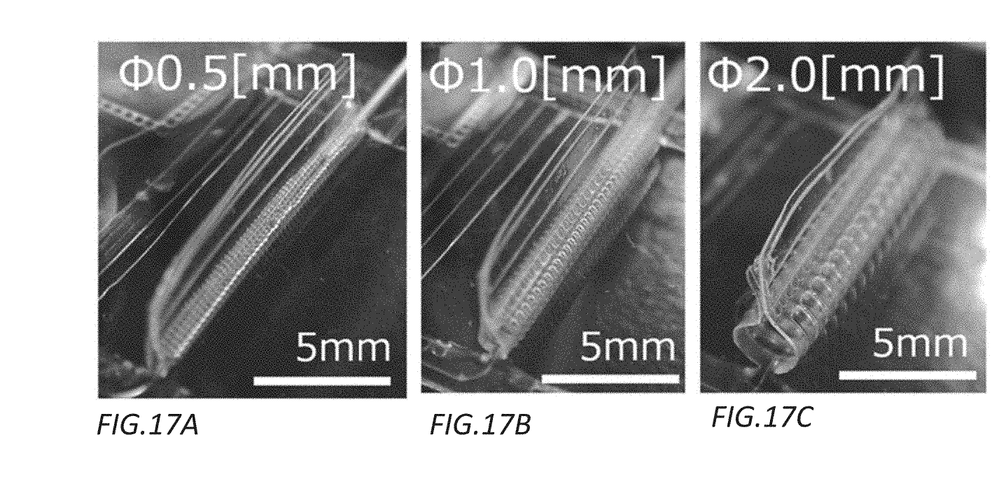

FIGS. 17A, 17B and 17C shows images of the plurality of types of culture substrates having different inner diameters, and balloon actuators. FIG. 17A shows a culture substrate having an inner diameter of about 0.5 mm, FIG.17B shows a culture substrate having an inner diameter of about 1.0 mm, and FIG. 17C shows a culture substrate having an inner diameter of about 2.0 mm.

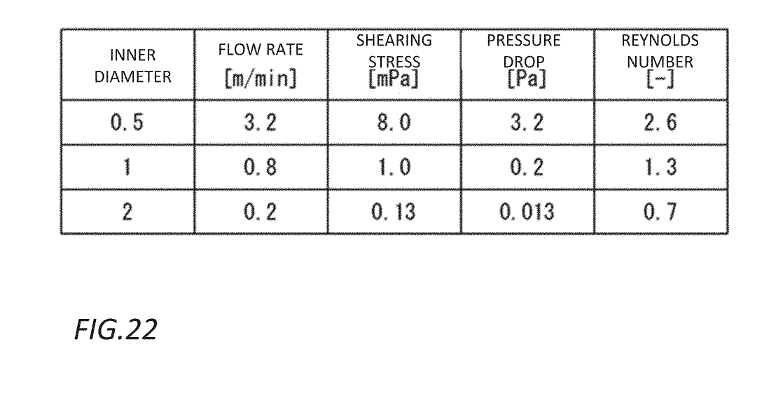

FIG. 22 is a table showing the relationship between the inner diameter of the tubular-form culture substrate (hereinafter also simply referred to as “tube”) and the dynamic characteristics of the fluid that flows in the tube. In this table, flow rate u, shearing stress τ, pressure drop ΔP, and Reynolds number Re are calculated according to the following formulae (1) to (4).

u=Q/(πr²) (1)

τ=4μQ/(πr³) (2)

ΔP=8μQL/(πr⁴) (3)

Re=2ρQ/(μπr) (4)

where Q is the flow rate of the liquid, μ is the viscosity of the liquid, ρ is the density of the liquid, L is the length of the tube, and r is the radius of the tube. The flow rate Q of the liquid is 0.05 mL/min, and the liquid was perfused for 3 minutes.

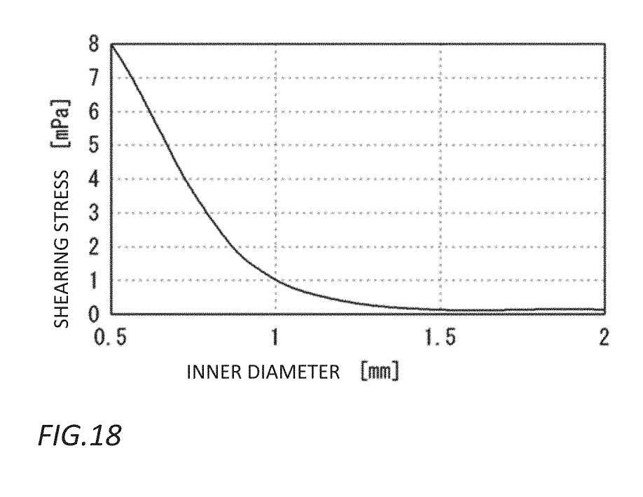

It is understood from formula (2) that the shearing stress τ changes when the radius r (inner diameter 2r) of the tube is changed. FIG. 18 shows the relationship between the inner diameter of the tube and the shearing stress.

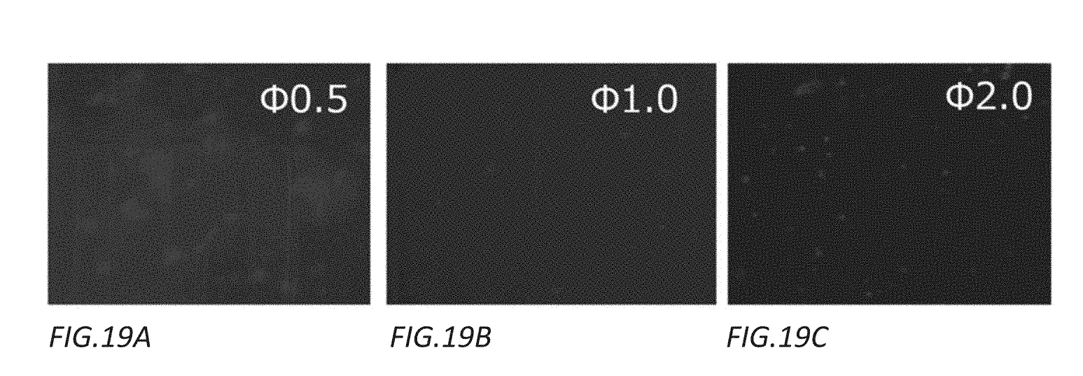

FIGS. 19A, 19B and 19C show images obtained by causing a fluid containing Texas Red as a model of a lipophilic drug to flow in three types of tubular-form culture substrates having different diameters, and imaging fluorescence signals from the Texas Red. FIG. 19A shows a case where the inner diameter is 0.5 mm, FIG. 19B shows a case where the inner diameter is 1.0 mm, and FIG. 19C shows a case where the inner diameter is 2.0 mm. When the case where the inner diameter of the tubular-form culture substrate is 0.5 mm is compared with the case where the inner diameter thereof is 1.0 mm, the image is redder in the former case than in the latter case. When the case where the inner diameter of the tubular-form culture substrate is 1.0 mm is compared with the case where the inner diameter thereof is 2.0 mm, the image is redder in the former case than in the latter case. Therefore, the smaller the inner diameter of the tube is, the faster the lipophilic drug is absorbed.

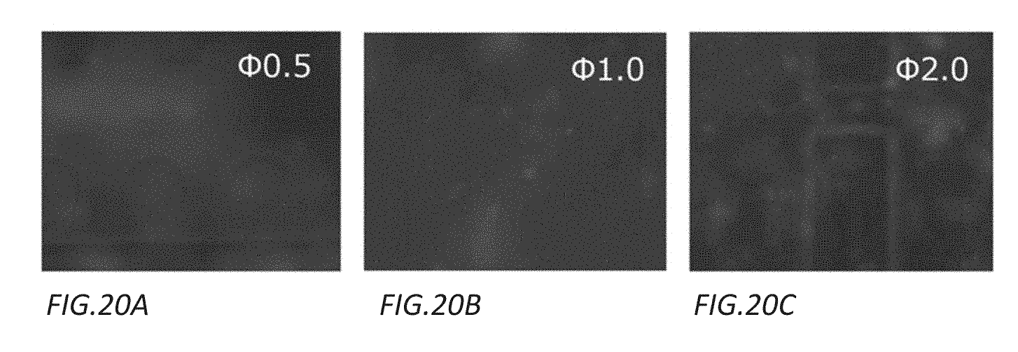

FIGS. 20A, 20B and 20C show images obtained by causing a fluid containing calcein as a model of a hydrophilic drug to flow in three types of tubular-form culture substrates having different inner diameters, and imaging fluorescence signals from the calcein. FIG. 20A shows a case where the inner diameter is 0.5 mm, FIG. 20B shows a case where the inner diameter is 1.0 mm, and FIG. 20C shows a case where the inner diameter is 2.0 mm. In any of the tubular-form culture substrates having the different inner diameters, the state of the stained caco-2 cells remained unchanged, and substantially no difference was observed in the speed of absorbing the hydrophilic drug.

The above results lead to the following considerations.

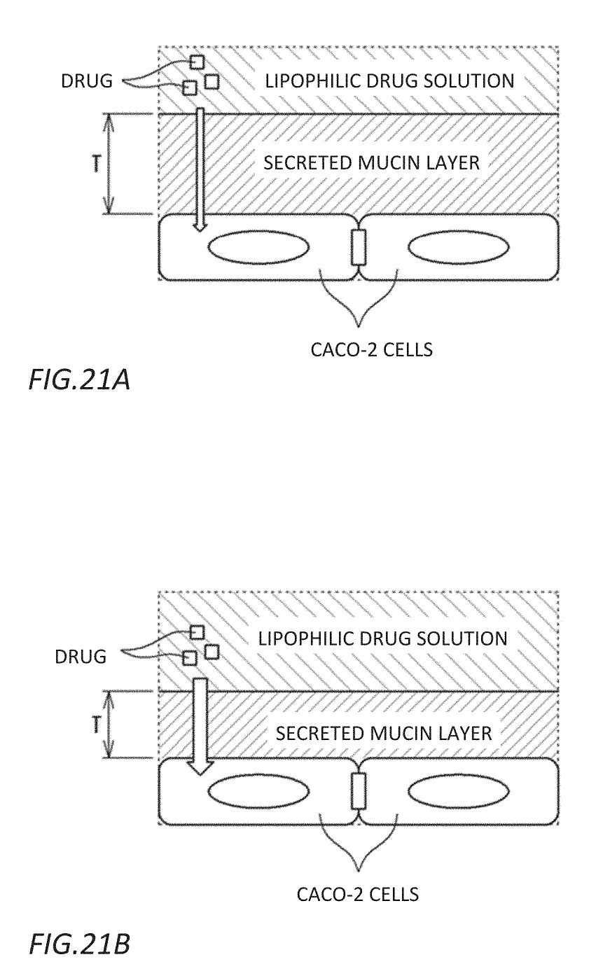

As shown in FIGS. 21A and 21B, highly viscous mucin is secreted from the caco-2 cells cultured on the culture substrate, and the surface of the caco-2 cells is coated with a hydrophilic mucin layer (mucous layer). This mucin layer is also called a non-agitated water layer, and serves as a barrier interfering with absorption of the lipophilic drug into the caco-2 cells, while does not interfere with absorption of the hydrophilic drug into the caco-2 cells. In addition, it is considered that a thickness T of the mucin layer is varied by a shearing stress that acts on the inner surface of the culture substrate due to flow of the fluid. As shown in FIG. 22, the greater the inner diameter r of the tubular-form culture substrate is, the smaller the shearing stress τ is; whereas the smaller the inner diameter r is, the greater the shearing stress τ is. Accordingly, it can be considered that the greater the inner diameter r is, the greater the thickness T of the mucin layer is, which decreases the speed of absorption of the lipophilic drug (refer to FIG. 21A); whereas the smaller the inner diameter r is, the smaller the thickness T of the mucin layer is, which increases the speed of absorption of the lipophilic drug (refer to FIG. 21B). Therefore, the speed of absorption of the lipophilic drug can be controlled by controlling the shearing stress by adjusting the inner diameter of the tubular-form culture substrate. Further, it can be considered that, by adjusting the inner diameter of the tubular-form culture substrate, various flow conditions in canals of a living body can be reproduced, and the speed of absorption of the lipophilic drug can also be reproduced.

### REFERENCE SIGNS LIST

10 cell culture apparatus

11 culture substrate

11 a culture surface

12 drive unit

30 balloon actuator

30a both end portions

53 introduction tube

S cell

1. A cell culture apparatus comprising:
a culture substrate having a culture surface on which cells are cultured; and
a drive unit configured to allow opening and closing of the culture substrate between a closed form and an open form, wherein
the closed form is a form in which the culture substrate forms a flow path having an internal volume with the culture surface being an internal surface of the flow path, and
the open form is a form in which the culture surface of the culture substrate is opened outward more than the culture surface of the culture substrate in the closed form.

2. The cell culture apparatus according to claim 1, wherein the culture substrate in the open form is flat in shape.

3. The cell culture apparatus according to claim 1, wherein the culture substrate in the closed form is tubular in shape.

4. The cell culture apparatus according to claim 1, wherein the drive unit has a balloon actuator provided on a surface, of the culture substrate, on a side opposite to the culture surface.

5. The cell culture apparatus according to claim 4, wherein the balloon actuator has a region through which a liquid having transmitted through the culture substrate passes.

6. The cell culture apparatus according to claim 4, wherein
the culture substrate in the closed form is tubular in shape, and
in the culture substrate in the closed form, culture-substrate-side surfaces of both end portions, opposing each other, of the balloon actuator are seal surfaces that come into surface-contact with each other.

7. The cell culture apparatus according to claim 6, wherein hydrophobic treatment is applied to the seal surfaces.

8. The cell culture apparatus according to claim 1, wherein hydrophobic treatment is applied to an inner peripheral surface of an end portion of the culture substrate in the closed form, to which an introduction tube for causing a fluid to flow into the culture substrate is connected.
